(19) United States
(12) Patent Application Publication
(10) Pub. No.: US 2018/0038866 A1
(43) Pub. Date: Feb. 8, 2018

(54) APPARATUS AND METHOD FOR DETERMINING PROGNOSIS OF BREAST CANCER AND WHETHER TO USE CHEMOTHERAPY

(71) Applicant: INDUSTRY-ACADEMIC COOPERATION FOUNDATION, YONSEI UNIVERSITY, Seoul (KR)

(72) Inventor: Soon Myung PAIK, Seoul (KR)

(73) Assignee: INDUSTRY-ACADEMIC COOPERATION FOUNDATION, YONSEI UNIVERSITY, Seoul (KR)

(21) Appl. No.: 15/553,757

(22) PCT Filed: Jan. 6, 2016
(86) PCT No.: PCT/KR2016/000084

§ 371 Date: Aug. 25, 2017

(30) Foreign Application Priority Data
Feb. 27, 2015 (KR) 10-2015-0028232

(51) Int. Cl. G01N 33/574; C12Q 1/68

(52) U.S. Cl. CPC G01N 33/57415; C12Q 1/6886; G01N 2800/52; C12Q 2600/118; C12Q 2600/158

(57) ABSTRACT

The present invention relates to an apparatus and a method for determining breast cancer prognosis and whether to use chemotherapy, and more specifically, to an apparatus and method capable of predicting prognosis and chemotherapeutic effects, which are necessary in determining a treatment plan for a breast cancer patient, by use of progesterone receptor immunostaining results and Ki67 protein immunostaining results. The method for determining breast cancer prognosis according to the present invention can be performed at low costs compared to a conventional method for determining breast cancer prognosis, and can provide a standardized index in predicting breast cancer prognosis and chemotherapeutic effects, due to its relatively high accuracy, and ultimately can contribute to improving national health and welfare.

### TECHNICAL FIELD

The present invention relates to an apparatus and a method for determining breast cancer prognosis and whether to use chemotherapy, and more particularly, to an apparatus and method capable of predicting prognosis and chemotherapeutic effects, which are necessary in determining a treatment plan for a breast cancer patient, based on the results of immunostaining of progesterone receptor and the results of immunostaining of Ki67 protein.

### BACKGROUND ART

Estrogen hormone receptor positive (ER+) breast cancer patients free of axillary lymph node metastasis, who account for about half of breast cancer patients, have a 10-year recurrence rate of 15% when receive five years of anti-hormone therapy alone, and show a decreased absolute value of 10-year recurrence rate of about 5% when receive additional chemotherapy (Fisher et al, Lancet. 10; 364 (9437):858-68, PMID: 15351193). However, the Oncotype Dx test developed in 2004 revealed that chemotherapy is required only in some patients (Paik et al, J Clin Oncol 24(23):3726-34, PMID: 16720680).

Since the Oncotype Dx test was developed, many tests for predicting breast cancer prognosis based on transcription factor analysis, including Mammaprint, Endopredict, Breast Cancer Index, Prosigna and the like, have been developed and commercialized. However, the Oncotype Dx test is the only test with proven clinical applicability, which determines whether to use chemotherapy (Paik et al, J Clin Oncol 24(23):3726-34, PMID: 16720680).

Meta-analysis revealed that tests for predicting breast cancer prognosis based on transcription factor analysis, such as Oncotype Dx or Mammaprint, all classify a breast cancer with low growth rate among ER+ breast cancers as a cancer with good prognosis. Thus, it can be considered that all tests that theoretically measure growth rate are interchangeable with Oncotype Dx (Wirapati et al, Breast Cancer Research. 2008; 10(4):R65. doi: 10.1186/bcr2124. PMID: 18662380).

However, in actual clinical application to individual patients, tests are performed using different gene expression analysis methods. Furthermore, all transcription factors are not analyzed, but only certain genes are analyzed, and for this reason, a step of normalization based on the expression level of a control gene in a sample is performed, and thus results different from those of meta-analysis appear, and the rate of concordance with Oncotype Dx is low. For example, EndoPredict was 76% concordant with the risk category of Oncotype Dx (Varga et al, PLoS One. 2013; 8(3):e58483, PMID: 23505515). Nevertheless, several transcription factor analysis methods as described above have been already used in various countries in the world, including Korea, and have the problem of being expensive, like Oncotype Dx.

Accordingly, the present inventors have found an apparatus and a method for determining breast cancer prognosis and whether to use chemotherapy, which can predict the risk category of Oncotype Dx with an accuracy of 75% or higher in an inexpensive manner by the use of the Ki67 protein which is representative of growth-related proteins, thereby reaching the present invention.

### DISCLOSURE

### Technical Problem

It is an object of the present invention to provide an apparatus capable of determining breast cancer prognosis and whether to use chemotherapy, in an inexpensive manner with high accuracy corresponding to an at least 75% concordance with the risk category of Oncotype Dx.

Another object of the present invention is to provide a method capable of determining breast cancer prognosis and whether to use chemotherapy, in an inexpensive manner with high accuracy corresponding to an at least 75% concordance with the risk category of Oncotype Dx.

### Technical Solution

In the prior art, the Oncotype Dx test was proposed as a prognosis determination method for selecting a subsequent method for treating cancer in breast cancer patients, but had a problem in that it is very expensive, and thus is applied only to a very limited number of patients.

The Oncotype Dx measures the expression of 21 genes by reverse transcription polymerase chain reaction and yields a recurrence score (RS) between 1 and 100. A RS lower than 18 is classified as a low-risk group, and it was verified that the low-risk group has a 10-year recurrence rate of less than 10% even when treated with hormonal therapy alone without using chemotherapy. On the other hand, it was verified that a high-risk group with a RS higher than 30 has a very high recurrence rate, but the therapeutic effects of anticancer agents in this group are very great. An intermediate-risk group with an RS of 18 to 30 shows little or no anticancer effects and has a recurrence rate of 10% or more, and thus there is still no distinct treatment plan for this group (Paik et al, J Clin Oncol 24(23):3726-34, PMID: 16720680). This intermediate-risk group gives common patients the option of a treatment plan, and it is known that about half of the patients opt for chemotherapy, and a treatment plan for these patients will be determined in the future according to the results of TAILORx (the Trial Assigning IndividuaLized Options for Treatment Rx) clinical trial (Sparano et al N Engl J Med. 2015 Sep. 27. [Epub ahead of print] PubMed PMID: 26412349).

In the TAILORx clinical trial, a RS lower than 11 is classified as a low-risk group who received no chemotherapy, and a RS of 11 or higher but lower than 25 was classified as an intermediate-risk group who was subjected to randomized clinical trials, and a RS of 25 or higher was classified as a high-risk group who received chemotherapy. Although clinical trial results for the intermediate-risk group have not yet been reported, the New England Journal of Medicine recently announced that low-risk group patients with a RS lower than 11 had a very good prognosis even when received hormonal therapy alone (Sparano et al, N Engl J Med. 2015 Sep. 27. [Epub ahead of print] PubMed PMID: 26412349). Thus, classifying the Oncotype Dx recurrence score as shown in Table 1 below is clinically very significant.

TABLE 1

| Oncotype Dx | | |
|---|---|---|
| recurrence | TAILORx-based risk | |
| score (RS) | classification | Treatment method |
| <11 | 1. TAILORx low-risk | Not use chemotherapy |
| 11 to 17 | 2. Low risk (TAILORx | |
| | intermediate-risk) | |
| 18 to 24 | 3. TAILORx intermediate- | Selective chemotherapy |
| | risk | while watching for |
| | | TAILORx results |
| 25 to 30 | 4. Intermediate risk | Chemotherapy |
| | (TAILORx high-risk) | |
| >30 | 5. High risk | |

Oncotype Dx is currently provided under national health insurance in highly developed countries, including the USA, Ireland, Israel and the like, and is applied to 70,000 patients each year. However, in Korea, Oncotype Dx is not covered under insurance, is very expensive (at least 4 million won (Korean currency), and for this reason, is applied only to a very limited number of patients.

Meanwhile, the most important genes of 21 Oncotype Dx genes are ESR1, PGR, BCL2 and SCUBE2 in the estrogen receptor group, and MKI67, STK15, Survivin, CCNB1 and MYBL2 in the proliferation group (Paik et al, N Eng J Med, 351(27):2817-26, PMID 15591335). Thus, progesterone receptor, which is representative of the estrogen receptor group, MKI67 (i.e., Ki67 protein) which is representative of the proliferation group, are likely to be replaced by immunostaining, and many researchers attempted this replacement and announced papers. However, it has not yet been verified that Ki67 can predict the therapeutic effect of chemotherapy in the NSABP B-20 clinical trial.

If the Oncotype Dx result that is the only marker proved to enable prediction of the therapeutic effect of chemotherapy can be predicted using the Ki67 protein, clinical application of the Ki67 protein is possible. However, currently, the reason why the Oncotype Dx result cannot be accurately predicted using the Ki67 protein is because evaluation criteria for immunostaining are not standardized. Recently, in six famous cancer hospitals, the Ring study was performed using tissues of the same patients, and in two of the six hospitals, the pathologists performed visual microscopic observation. The results of the observation are graphically shown in FIG. 1. As can be seen in FIG. 1, the results are not consistent with each other (Polley et al, J Natl Cancer Inst. 2013 Dec. 18; 105 (24):1897-906. doi: 10.1093/jnci/djt306. Epub 2013 Nov. 7. PubMed PMID: 24203987).

Despite this limitation, some researchers attempted to make an algorithm that predicts the Oncotype Dx result using several markers including the Ki67 protein, but this algorithm showed an excessively low accuracy when applied in clinical practice. For example, Allison et al. could predict the Oncotype Dx result with an accuracy of about 32% by tree-type classification (Allison et al, Breast Cancer Res Treat, 2012; 131(2):413-24, PMID 21369717). Furthermore, Kelin et al. developed a recurrence score algorithm as shown in the following equation (1) and attempted to predict Oncotype Dx recurrence score, but the rate of concordance with Oncotype Dx risk classification was only about 55% (Klein et al, Modern Pathology 2013; 26(5):658-64, PMID:23503643).

Recurrence score=15.31385+Nottingham score*1.4055+ERIHC*(0.01924)+PRIHC*(0.02925)±(0 for HER2 negative, 0.77681 for equivocal, 11.58134 for HER2 positive)+tumor size*0.78677+Ki-67 index*0.13269 Equation 1

Accordingly, the present inventors have found that the use of immunostaining results for progesterone receptor and immunostaining results for Ki67 protein makes it possible to predict Oncotype Dx results and breast cancer prognosis with high accuracy in a very simple and inexpensive manner, thereby reaching the present invention.

Specifically, the present invention provides an apparatus and a method for determining breast cancer prognosis and whether to use chemotherapy, which comprise: extracting a breast tissue sample from a patient; and determining the risk of recurrence score based on the percentage of the number of cancer cells including stained progesterone receptor relative to the total number of cancer cells or an Allred score after immunostaining of progesterone receptor in the breast tissue sample, and based on the percentage of cancer cells including stained Ki67 protein relative to the total number of cancer cells after immunostaining of Ki67 protein in the breast tissue sample.

Specifically, in accordance with one embodiment of the present invention, there is provided an apparatus for determining breast cancer prognosis and whether to use chemotherapy, the apparatus comprising:

a first input unit configured to receive the percentage of the number of cancer cells including stained progesterone receptor relative to the total number of cancer cells or an Allred score, after immunostaining of progesterone receptor in a breast tissue sample extracted from a breast cancer patient;

a second input unit configured to receive the percentage of the number of cancer cells including stained Ki67 protein relative to the total number of cancer cells, after immunostaining of Ki67 protein in the breast tissue sample;

a classification calculation unit configured to classify the breast cancer patient as low risk when the percentage of the number of cancer cells including stained progesterone receptor relative to the total number of cancer cells, inputted in the first input unit, is higher than 20%, or the Allred score is 5 or higher, and when the percentage of the number of cancer cells including stained Ki67 protein relative to the total number of cancer cells, inputted in the second input unit, is lower than 20%; and

an output unit configured to output analysis results from the classification calculation unit.

In accordance with another embodiment of the present invention, there is provided an apparatus for determining breast cancer prognosis and whether to use chemotherapy, the apparatus comprising:

a first input unit configured to receive the percentage of the number of cancer cells including stained progesterone receptor relative to the total number of cancer cells or an Allred score, after immunostaining of progesterone receptor in a breast tissue sample extracted from a breast cancer patient;

a second input unit configured to receive the percentage of the number of cancer cells including stained Ki67 protein relative to the total number of cancer cells, after immunostaining of Ki67 protein in the breast tissue sample;

a classification calculation unit configured to classify the breast cancer patient as low risk when the percentage of the number of cancer cells including stained progesterone receptor relative to the total number of cancer cells, inputted in the first input unit, is 20% or lower, or the Allred score is lower than 5, and when the percentage of the number of cancer cells including stained Ki67 protein relative to the total number of cancer cells, inputted in the second input unit, is lower than 10%; and

an output unit configured to output analysis results from the classification calculation unit.

In accordance with still another embodiment of the present invention, there is provided a method for providing information for determining breast cancer prognosis and whether to use chemotherapy, the method comprising classifying a breast cancer patient as low risk when the percentage of the number of cancer cells including stained progesterone receptor relative to the total number of cancer cells, after immunostaining of progesterone receptor in a breast tissue sample extracted from the breast cancer patient, is higher than 20%, or an Allred score after immunostaining of progesterone receptor in the breast tissue sample is 5 or higher, and when the percentage of the number of cancer cells including stained Ki67 protein relative to the total number of cancer cells, after immunostaining of Ki67 protein, is lower than 20%.

In accordance with yet another embodiment of the present invention, there is provided a method for providing information for determining breast cancer prognosis and whether to use chemotherapy, the method comprising classifying a breast cancer patient as low risk when the percentage of the number of cancer cells including stained progesterone receptor relative to the total number of cancer cells, after immunostaining of progesterone receptor in a breast tissue sample extracted from the breast cancer patient, is 20% or lower, or an Allred score after immunostaining of progesterone receptor in the breast tissue sample is lower than 5, and when the percentage of the number of cancer cells including stained Ki67 protein relative to the total number of cancer cells, after immunostaining of Ki67 protein in the breast tissue sample, is lower than 10%.

According to one preferred embodiment of the present invention, the accuracy of risk classification can be increased either by measuring the percentage of the number of cancer cells including stained progesterone receptor or Ki67 protein relative to the total number of cancer cells in a hot spot with the highest staining index, after immunostaining of progesterone receptor or Ki67 protein in a breast tissue sample extracted from a breast cancer patient, or by measuring the Allred score.

More preferably, when the percentage of the number of cancer cells including stained progesterone receptor relative to the total number of cancer cells, after immunostaining of progesterone receptor in a breast tissue sample extracted from a breast cancer patient, is 20% or lower, or when an Allred score after immunostaining of progesterone receptor in the breast tissue sample is lower than 5, the percentage of the number of cancer cells including stained Ki67 protein relative to the total number of cancer cells in a hot spot with the highest staining index after immunostaining of Ki67 protein may be measured.

Herein, although the shape or area of the hot spot with the highest staining index is not limited, the hot spot may be, for example, a circular spot having a radius of 400 to 650 μm.

In the present invention, the method for measuring the number of cancer cells including stained progesterone receptor or stained Ki67 protein relative to the total number of cancer cells is not particularly limited, but the number of the cancer cells can be visually measured using a 400× microscope.

Furthermore, the low risk may mean a group corresponding to a recurrence score (RS) of 0 to 30, preferably 0 to 24, more preferably 0 to 18, as determined according to conventional Oncotype Dx.

In the present invention, “immunostaining” means immunohistochemical staining that stains a certain substance in tissue or cells with a labeled antibody based on an antigen-antibody reaction. More specifically, immunostaining is performed using an enzyme or a fluorescent substance as a marker so that the presence or absence of a specific substance in cells can be visually detected. First, a labeled or non-labeled primary antibody specific for a substance to be detected is used, and a marker-conjugated secondary antibody or polymer capable of binding to the primary antibody is used. Next, the presence or absence and amount of the substance to be detected can be determined by measuring the presence or absence and intensity of the marker. In the present invention, immunostaining may be performed using a primary antibody specific for progesterone receptor (PR) or Ki67 protein and a second antibody or polymer labeled with a fluorescent substance or enzyme. For example, immunostaining of the progesterone receptor may be performed using a Ventana XT Instrument (Ventana Japan, Tokyo, Japan), but is not limited thereto. Immunostaining of the Ki67 protein may be performed using anti-Ki67 protein antibody (Abcam, Mass., USA), but is not limited thereto. In addition, any progesterone receptor-specific antibody or any Ki67 protein-specific antibody, which may be used in immunohistochemical staining in the art, may be used without limitation.

In the present invention, the “Allred score” is a scoring system that determines how strongly a hormone receptor (progesterone receptor in the present invention) in breast cancer tissue appears (see Harvey et al. Journal of Clinical Oncology, 17, 1474[1999]). More specifically, after immunohistochemical staining of progesterone receptor, staining intensity (score: 0 to 3) and stained percentage (score: 0 to 5) are expressed as numerical values and summed to obtain a total score. Evaluation criteria for staining intensity score and staining percentage score are as follows:

(1) Staining intensity score

0: negative; 1: weak; 2: intermediate; 3: strong.

(2) Staining percentage score

0: the percentage of stained cancer cells is 0%; 1: the percentage of stained cancer cells is higher than 0% but not higher than 1%; 2: the percentage of stained cancer cells is 1% to 10%; 3: the percentage of stained cancer cells is 11% to 33%; 4: the percentage of stained cancer cells is 34% to 66%; 5: the percentage of stained cancer cells is 67% to 100%.

Herein, “the percentage of stained cancer cells” means the number of cancer cells including stained progesterone receptor relative to the total number of cancer cells in a sample in the results of immunohistochemical staining of progesterone receptor.

### Advantageous Effects

An apparatus and a method for determining breast cancer prognosis and whether to use chemotherapy according to the present invention can be performed at low costs compared to a conventional method for determining breast cancer prognosis and whether to use chemotherapy, and show relatively high accuracy. Thus, the apparatus and method of the present invention can provide a standardized index in determining breast cancer prognosis and whether to use chemotherapy, and ultimately contribute to promoting national health and welfare.

### DESCRIPTION OF DRAWINGS

FIG. 1 is a figure excerpted from the paper of Polley et al. and is a relation view graphically showing the results obtained by visually and microscopically observing the results of Ki67 immunostaining by pathologists in any two hospitals (Polley et al, J Natl Cancer Inst. 2013 Dec. 18; 105(24):1897-906. doi: 10.1093/jnci/djt306. Epub 2013 Nov. 7. PubMed PMID: 24203987).

FIG. 2 is a graph obtained in Experimental Example 1 by immunostaining Ki67 protein, selecting as the X-axis variable the percentage of the number of cancer cells including stained Ki67 protein relative to the total number of cancer cells, immunostaining progesterone receptor, selecting as the Y-axis variable the percentage of the number of cancer cells including stained progesterone receptor relative to the total number of cancer cells, and then plotting the percentages according to different Oncotype Dx RS ranges.

FIG. 3A is a photograph showing the results of observing immunostaining results with a 400× microscope for any spot excluding a hot spot after Ki67 immunostaining for a breast tissue sample extracted from a breast cancer patient in Experimental Example 1.

FIG. 3B is a photograph showing the results of observing immunostaining results with a 400× microscope for a hot spot with the highest staining index after Ki67 immunostaining for a breast tissue sample extracted from a breast cancer patient in Experimental Example 1.

FIG. 4 is a graph obtained in Experimental Example 1 by immunostaining Ki67 protein in a hot spot, selecting as the X-axis variable the percentage of the number of cancer cells including stained Ki67 protein relative to the total number of cancer cells in a hot spot, immunostaining progesterone receptor, selecting as the Y-axis variable the percentage of the number of cancer cells including stained progesterone receptor relative to the total number of cancer cells, and then plotting the percentages according to different Oncotype Dx RS ranges.

FIGS. 5A and 5B are photographs showing the results of observing immunostaining results with a 400× microscope after Ki67 immunostaining for one patient in whom the percentage of the number of stained cancer cells after of immunostaining of progesterone receptor as shown in FIG. 4 is 20% or lower and the percentage of the number of stained cancer cells after immunostaining of Ki67 is lower than 10% and who is indicated by symbol ▴. In FIG. 5A, cancer cells were Ki67 stained, but in FIG. 5B, immune cells other than cancer cells were stained.

FIG. 6 shows the results of performing ROC analysis for classification results obtained by the method of the present invention in Experimental Example 1.

FIG. 7 is a graph comparing classification results obtained by the method of the present invention with TAILORx-based Oncotype Dx classification results in order to predict the effect of classification results obtained by the method of the present invention on clinical practice in Experimental Example 1.

FIG. 8 is a graph comparing classification results obtained by the method of the present invention with Oncotype Dx classification results in order to predict the effect of classification results obtained by the method of the present invention on clinical practice in Experimental Example 1.

FIG. 9 is a graph obtained in Experimental Example 2 by immunostaining Ki67 protein, selecting as the X-axis variable the percentage of the number of cancer cells including stained Ki67 protein relative to the total number of cancer cells, immunostaining progesterone receptor, selecting as the Y-axis variable an Allred score after immunostaining of progesterone receptor, and then plotting the percentage and the Allred score according to different Oncotype Dx RS ranges.

FIG. 10 is a graph obtained in Experimental Example 2 by immunostaining Ki67 protein, selecting as the X-axis variable the percentage of the number of cancer cells including stained Ki67 protein relative to the total number of cancer cells in a hot spot, immunostaining progesterone receptor, selecting as the Y-axis variable an Allred score after immunostaining of progesterone receptor, and then plotting the percentage and the Allred score according to different Oncotype Dx RS ranges.

FIG. 11 shows the results of performing ROC analysis for classification results obtained by the method of the present invention in Experimental Example 2.

FIG. 12 is a graph comparing classification results obtained by the method of the present invention with TAILORx-based Oncotype Dx classification results in order to predict the effect of classification results obtained by the method of the present invention on clinical practice in Experimental Example 2.

FIG. 13 is a graph comparing classification results obtained by the method of the present invention with Oncotype Dx classification results in order to predict the effect of classification results obtained by the method of the present invention on clinical practice in Experimental Example 2.

FIG. 14 is a graph obtained in Experimental Example 3 by immunostaining Ki67 protein, selecting as the X-axis variable the percentage of the number of cancer cells including stained Ki67 protein relative to the total number of cancer cells, immunostaining progesterone receptor, selecting the Y-axis variable the percentage of the number of cancer cells including stained progesterone receptor relative to the total number of cancer cells, and then plotting the percentages according to different Oncotype Dx RS ranges.

FIG. 15 is a graph obtained in Experimental Example 3 by immunostaining Ki67 protein in a hot spot, selecting as the X-axis variable the percentage of the number of cancer cells including stained Ki67 protein relative to the total number of cancer cells, immunostaining progesterone receptor, selecting as the Y-axis variable the percentage of the number of cancer cells including stained progesterone receptor relative to the total number of cancer cells, and then plotting the percentages according to different Oncotype Dx RS ranges.

FIG. 16 shows the results of performing ROC analysis for classification results obtained by the method of the present invention in Experimental Example 3.

FIG. 17 is a graph comparing classification results obtained by the method of the present invention with TAILORx-based Oncotype Dx classification results in order to predict the effect of classification results obtained by the method of the present invention on clinical practice in Experimental Example 3.

FIG. 18 is a graph comparing classification results obtained by the method of the present invention with Oncotype Dx classification results in order to predict the effect of classification results obtained by the method of the present invention on clinical practice in Experimental Example 3.

FIG. 19 is a graph obtained in Experimental Example 4 by immunostaining Ki67 protein, selecting as the X-axis variable the percentage of the number of cancer cells including stained Ki67 protein relative to the total number of cancer cells, immunostaining progesterone receptor, selecting as the Y-axis variable an Allred score after immunostaining of progesterone receptor, and then plotting the percentage and the Allred score according to different Oncotype Dx RS ranges.

FIG. 20 is a graph obtained in Experimental Example 4 by immunostaining Ki67 protein in a hot spot, selecting as the X-axis variable the percentage of the number of cancer cells including stained Ki67 protein relative to the total number of cancer cells, immunostaining progesterone receptor, selecting as the Y-axis variable an Allred score after immunostaining of progesterone receptor, and then plotting the percentage and the Allred score according to different Oncotype Dx RS ranges.

FIG. 21 shows the results of performing ROC analysis for classification results obtained by the method of the present invention in Experimental Example 4.

FIG. 22 is a graph comparing classification results obtained by the method of the present invention with TAILORx-based Oncotype Dx classification results in order to predict the effect of classification results obtained by the method of the present invention on clinical practice in Experimental Example 4.

FIG. 23 is a graph comparing classification results obtained by the method of the present invention with Oncotype Dx classification results in order to predict the effect of classification results obtained by the method of the present invention on clinical practice in Experimental Example 4.

FIG. 24 is a graph obtained in Comparative Example 1 by immunostaining Ki67 protein in a patient, selecting as the X-axis variable the percentage of the number of cancer cells including stained Ki67 protein relative to the total number of cancer cells according to subjective judgment of a pathologist without performing image analysis, immunostaining progesterone receptor, selecting as the Y-axis variable the percentage of the number of cancer cells including stained progesterone receptor relative to the total number of cancer cells, and then plotting the percentages according to different Oncotype Dx RS ranges.

### BEST MODE

The present invention provides an apparatus and a method for determining breast cancer prognosis and whether to use chemotherapy, which comprise extracting a breast tissue sample from a patient, and then determining the risk of recurrence score based on either the percentage of the number of cancer cells including stained progesterone receptor relative to the total number of cancer cells or an Allred score, which results from immunostaining of progesterone receptor, and the percentage of the number of cancer cells including stained Ki67 protein relative to the total number of cancer cells, which results from immunostaining of Ki67 protein.

### MODE FOR INVENTION

Hereinafter, the present invention will be described in further detail with reference to examples. It will be obvious to those skilled in the art that these examples are for illustrative purposes only and are not intended to limit the scope of the present invention.

### EXAMPLES

### Experimental Example 1

In order to make an inexpensive test to be substituted for Oncotype Dx, image analysis for the results of progesterone and Ki67 immunostaining on 46 patients who underwent Oncotype Dx, among ER+/N− patients who received treatment in the breast cancer center of Yonsei Cancer Hospital, was performed using Image J program (http://imagej.nih.gov) and ImmunoRatio plug in (Tumoinet et al, Breast Cancer Res. 2010; 12(4):R56). However, ImmunoRatio used in this experiment uses a general image analysis method that can be understood by any person skilled in the art, and it can be replaced by another image analysis method or conventional program.

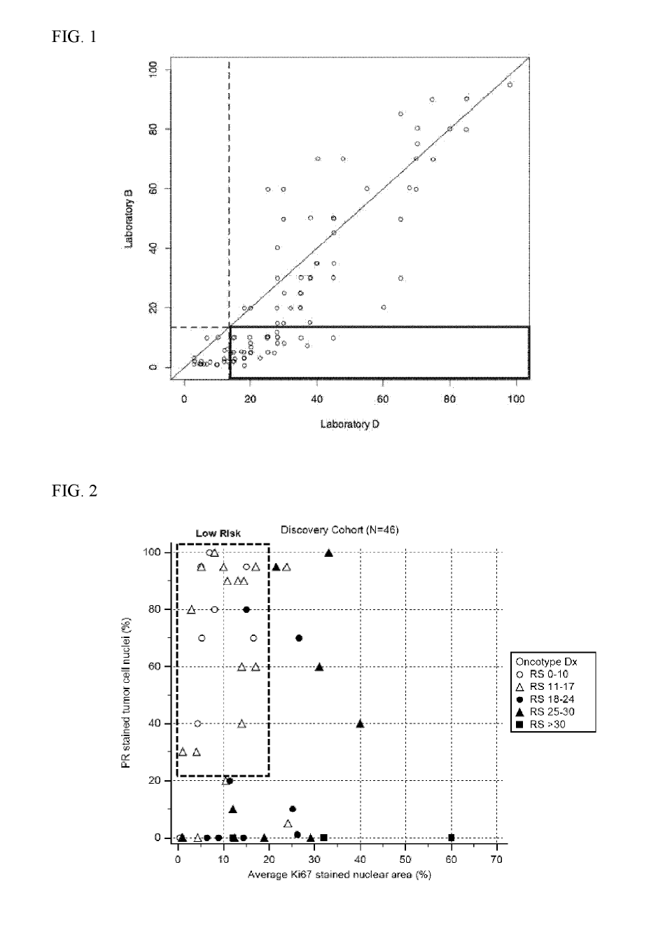

Specifically, Ki67 protein in each of breast tissue samples extracted from the breast cancer patients was immunostained, and the percentage of the number of cancer cells including stained Ki67 protein relative to the total number of cancer cells was selected as the X-axis variable (analysis of at least three 400× fields). Furthermore, progesterone receptor was immunostained, and the percentage of the number of cancer cells including stained progesterone receptor relative to the total number of cancer cells was selected as the Y-axis variable. The percentages were graphically plotted in FIG. 2 according to different Oncotype Dx recurrence score (RS) ranges of the patients.

As can be seen in FIG. 2, in cases in which the percentage of the number of cancer cells including stained progesterone receptor relative to the total number of cancer cells after immunostaining of progesterone receptor was higher than 20% and in which the percentage of the number of cancer cells including stained Ki67 protein relative to the total number of cancer cells after immunostaining of Ki67 protein was lower than 20%, all of these cases, excluding a first intermediate-risk case (), belong to the RS low-risk group.

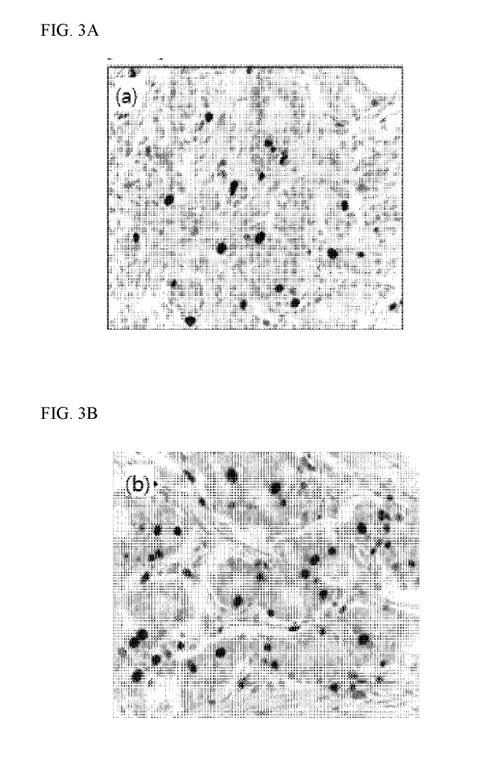

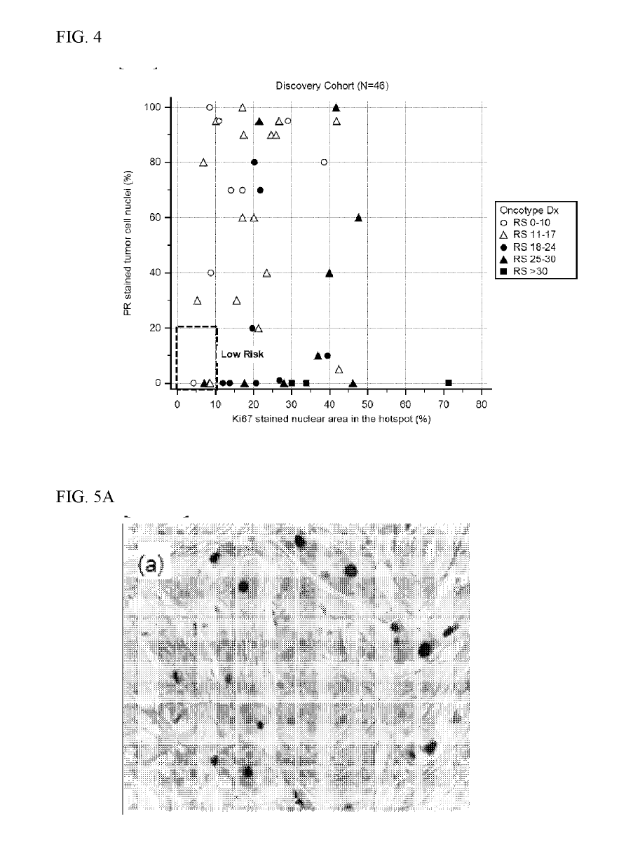

Furthermore, Ki67 protein in each of breast tissue samples extracted from the patients was immunostained, and then spots with the highest staining index were observed with a 400× microscope, and a circular spot having a radius of about 400 to 650 μm was selected as a hot spot. FIG. 3A is a photograph showing the results of observing any spot (a circular spot having a radius of 400 to 650 μm) excluding the hot spot with a 400× microscope, and FIG. 3B is a photograph showing the results of observing the hot spot with a 400× microscope. In the hot spot, the percentage of the number of cancer cells including stained Ki67 protein relative to the total number of cancer cells was selected as the X-axis variable, and the percentage of the number of cancer cells including stained progesterone receptor relative to the total number of cancer cells was selected as the Y-axis variable, and the percentages were graphically plotted in FIG. 4 according to different Oncotype Dx RS ranges of the breast cancer patients.

As can be seen in FIG. 4, in cases in which the percentage of the number of cancer cells including stained progesterone receptor relative to the total number of cancer cells after immunostaining of progesterone receptor was 20% or lower and in which the percentage of the number of cancer cells including stained Ki67 protein relative to the total number of cancer cells in the hot spot after immunostaining of Ki67 protein was lower than 10%, most of these cases belong to the RS low-risk group.

However, one patient showed a high RS value (▴), even though the percentage of cancer cells including stained progesterone receptor was 20% or lower and the percentage of cancer cells including stained Ki67 protein was lower than 10%. In this case, a pathologic slide review was performed, and as a result, there was severe lymphoid infiltrate. Namely, as can be seen in FIG. 5A, Ki67 staining in cancer cells was very low, whereas, as can be seen in FIG. 5B, many lymphocytes in the surrounding lymphoid infiltrate were stained. In other words, it appears that the RS value was wrongly classified as high risk.

Therefore, it can be seen that when the method of the present invention is used, the prognosis of breast cancer patients can be predicted in a very simple manner or with high accuracy, and particularly when the percentage of the number of stained cancer cells after immunostaining of progesterone receptor is 20% or lower, the accuracy of the prediction can further be increased by selecting and analyzing a hot spot.

Table 2 below compares the results of risk classification, performed using progesterone receptor immunostaining results and Ki67 immunostaining results based on image analysis as shown in FIGS. 2 and 4, with the results of Oncotype Dx classification.

TABLE 2

| | Oncotype Dx classification | | |
|---|---|---|---|
| Classification | | Intermediate to | |
| according to the | Low risk (RS | high risk (RS of | |
| present invention | lower than 18) | 18 or higher) | Sum |
| Low risk | 23 | 2 | 25 (54.3%) |
| Intermediate | 3 | 18 | 21 (45.7%) |
| to high risk | | | |
| Sum | 26 (56.5%) | 20 (43.5%) | 46 |

(p=0.00000082 (Fisher's exact test))

As can be seen in Table 2 above, the method of the present invention can relatively accurately identify low-risk patients who do not require chemotherapy. Since the low-risk group accounts for 50% or higher, many patients can be liberated from unnecessary chemotherapy, and thus the psychological anxiety of the patients and the physical burden and economic burden caused by chemotherapeutic side effects can be alleviated.

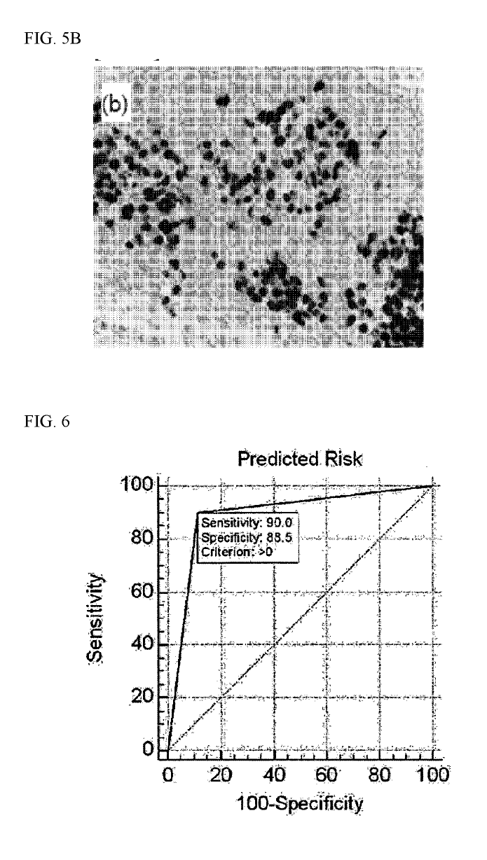

In addition, in order to confirm the statistical accuracy of the results, ROC (Receiver-Operating Characteristic) analysis was additionally performed. The results of the analysis are shown in Table 3 below and FIG. 6.

TABLE 3

| | | |
|---|---|---|
| Sensitivity | 90.00% | 68.30% to 98.77% |
| Specificity | 88.46% | 69.85% to 97.55% |
| Area Under Curve | 0.89 | 0.77 to 0.96 |
| Positive likelihood ratio | 7.8 | 2.66 to 22.84 |
| Negative likelihood ratio | 0.11 | 0.03 to 0.42 |
| Disease prevalence | 50.00% | |
| Positive predictive value | 88.64% | 68.71% to 97.91% |
| Negative predictive value | 89.84% | 69.84% to 98.46% |

As can be seen in Table 3 above and FIG. 6, sensitivity was 90.0% and specificity was 88.46%, suggesting that intermediate- or high-risk patients can be identified.

Furthermore, Table 4 below compares the results of risk classification, performed using progesterone receptor immunostaining results and Ki67 immunostaining results based on image analysis as shown in FIGS. 2 to 4, with the results of TAILORx-based Oncotype Dx classification.

TABLE 4

| | TAILORx-based Oncotype Dx classification | | | | | |
|---|---|---|---|---|---|---|
| Classification | | | RS higher | RS higher | | |
| according to the | | | than 18 but | than 24 but | | |
| present | RS lower | RS of 11 to | not higher | not higher | RS higher | |
| invention | than 11 | 18 | than 24 | than 30 | than 30 | Sum |
| Low risk | 8 (32%) | 15 (60%) | 1 (4%) | 1 (4%) | 0 | 25 (54.3%) |
| Intermediate to | 0 | 3 (14.3%) | 7 (33.3%) | 8 (38.1%) | 3 (14.3%) | 21 (45.7%) |
| high risk | | | | | | |
| Sum | 8 (17.4%) | 18 (39.1%) | 8 (17.4%) | 9 (19.6%) | 3 (6.5%) | 46 |

(p=0.00000082 (Fisher's exact test))

As can be seen in Table 4 above, the method of the present invention can very accurately identify low-risk patients who do not require chemotherapy.

Furthermore, in order to confirm the statistical significance of the experimental results, Chi-squared test was performed. As a result, as can be seen in Table 5 below, the results are statistically significant.

TABLE 5

| | |
|---|---|
| Chi-square | 28.814 |
| DF | 4 |
| Significance | P < 0.0001 |

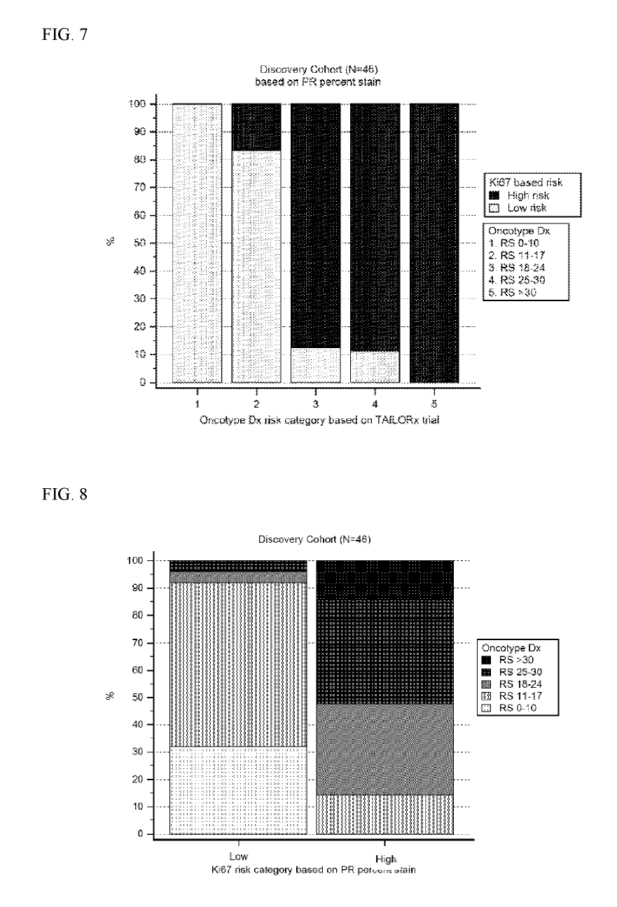

In addition, in order to predict the effect of the experimental results on clinical practice, the experimental results were graphically plotted in FIGS. 7 and 8. Patients (RS<11) classified as low-risk patients in the current TAILORx clinical trial, who receive no chemotherapy, were all classified as low risk by the method of the present invention, and 83.3% of patients with an RS of 11 to 18, generally classified as low risk, among the remaining patients, were classified as low risk by the method of the present invention. In addition, patients (RS>30) who must receive chemotherapy were all classified as high risk by the method of the present invention.

### Experimental Example 2

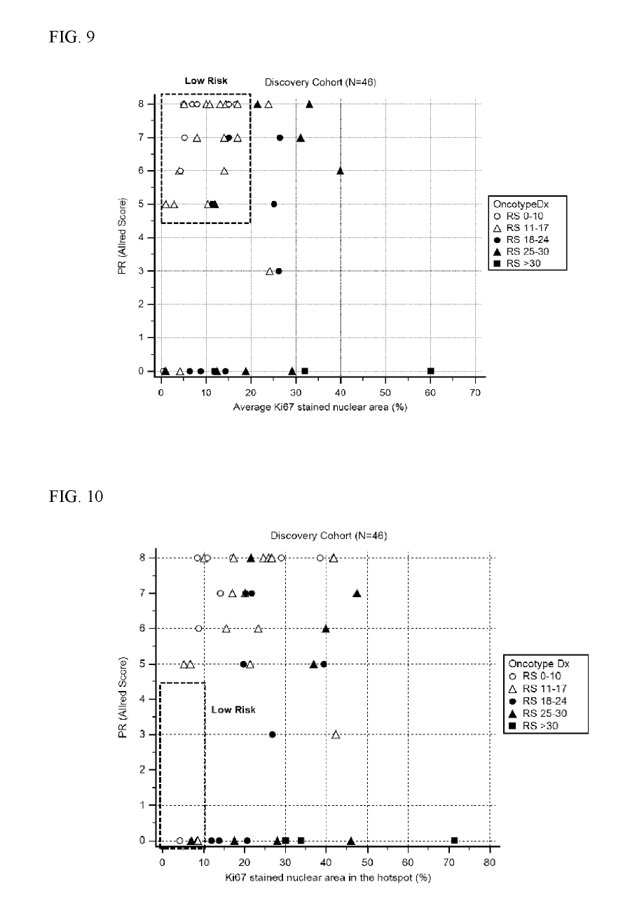

An Allred score after progesterone receptor immunostaining on 46 patients who underwent Oncotype Dx, among ER+/N− patients who received treatment in the breast cancer center of Yonsei Cancer Hospital, as described in Experimental Example 1 above, was measured and selected as the Y-axis variable. Furthermore, Ki67 protein in each of breast tissue samples extracted from the patients was immunostained, the percentage of the number of cancer cells including stained Ki67 protein relative to the total number of cancer cells was selected as the X-axis variable. Then, the Allred score and the percentage were graphically plotted in FIG. 9 according to different Oncotype Dx RS ranges of the breast cancer patients.

As can be seen in FIG. 9, in cases in which the Allred score after immunostaining of progesterone receptor was 5 or higher and in which the percentage of the number of cancer cells including stained Ki67 protein relative to the total number of cancer cells after immunostaining of Ki67 protein was 20% or lower, these cases mostly belong to the RS low-risk group.

Furthermore, Ki67 protein in each of breast tissue samples extracted from the patients was immunostained, and then a spot with the highest staining index was observed with a 400× microscope, and a circular spot having a radius of about 400 to 650 μm was selected as a hot spot. For the hot spot, the percentage of the number of cancer cells including stained Ki67 protein relative to the total number of cancer cells was selected as the X-axis variable, and the Allred score after immunostaining of progesterone receptor was selected as the Y-axis variable. Then, the percentage and the Allred score were graphically plotted in FIG. 10 according to different Oncotype Dx RS ranges of the patients.

As can be seen in FIG. 10, in cases in which the Allred score after immunostaining of progesterone receptor was lower than 5 and in which the percentage of the number of cancer cells including stained Ki67 protein relative to the total number of cancer cells in the hot spot after immunostaining of Ki67 protein was lower than 10%, these cases mostly belong to the RS low-risk group.

However, it was shown that one patient (▴) belongs to the high-risk group based on the RS value. In this case, a pathologic slide review was performed, and as a result, like Experimental Example 1, lymphoid infiltrates other than cancer cells were stained. Although the patient was classified as high risk in Oncotype Dx, the patient can be classified as low risk, because immunostaining results score only cancer cells.

Therefore, it can be seen that when the method of the present invention is used, the prognosis of breast cancer patients can be predicted using the Allred score and the results of Ki67 protein immunostaining in a very simple manner or with high accuracy, and particularly when the Allred score is lower than 5, the accuracy of the prediction can further be increased by selecting and analyzing a hot spot.

Table 6 below compares the results of risk classification as shown in FIGS. 9 and 10 with the results of Oncotype Dx classification.

TABLE 6

| | Oncotype Dx classification | | |
|---|---|---|---|
| Classification | | Intermediate to | |
| according to the | Low risk (RS | high risk (RS of | |
| present invention | lower than 18) | 18 or higher) | Sum |
| Low risk | 24 | 3 | 29 (63%) |
| Intermediate | 2 | 15 | 17 (37%) |
| to high risk | | | |
| Sum | 26 (56.5%) | 20 (43.5%) | 46 |

(p=0.000004738 (Fisher's exact test))

As can be seen in Table 6 above, the method of the present invention can relatively accurately identify low-risk patients who do not require chemotherapy. Since the low-risk group accounts for 50% or higher, many patients can be liberated from unnecessary chemotherapy, and thus the psychological anxiety of the patients and the physical burden and economic burden caused by chemotherapeutic side effects can be alleviated.

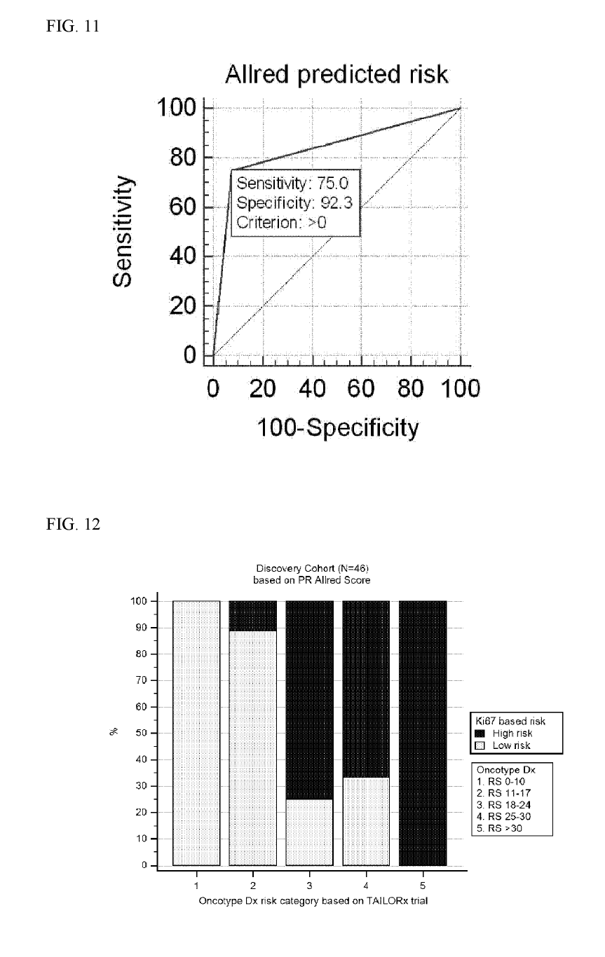

In addition, in order to confirm the statistical accuracy of the results, ROC (Receiver-Operating Characteristic) analysis was additionally performed. The results of the analysis are shown in Table 7 below and FIG. 11.

TABLE 7

| | | |
|---|---|---|
| Sensitivity | 75.00% | 50.90% to 91.34% |
| Specificity | 92.31% | 74.87% to 99.05% |
| Area Under Curve | 0.84 | 0.70 to 0.93 |
| Positive likelihood ratio | 9.75 | 2.51 to 37.81 |
| Negative likelihood ratio | 0.27 | 0.13 to 0.58 |
| Disease prevalence | 50.00% | |
| Positive predictive value | 90.70% | 68.46% to 99.07% |
| Negative predictive value | 78.69% | 58.75% to 91.97% |

As can be seen in Table 7 above and FIG. 11, sensitivity was 75% and specificity was 92.31%, suggesting that intermediate- or high-risk patients can be identified.

Furthermore, Table 8 below compares the risk classification results shown in FIGS. 9 and 10 with the results of TAILORx-based Oncotype Dx classification. Moreover, in order to confirm the statistical significance of the experimental results, Chi-squared test was performed, and the results are shown in Table 9 below.

TABLE 8

| | TAILORx-based Oncotype Dx classification | | | | | |
|---|---|---|---|---|---|---|
| Classification | | | RS higher | RS higher | | |
| according to the | | | than 18 but | than 24 but | | |
| present | RS lower | RS of 11 to | not higher | not higher | RS higher | |
| invention | than 11 | 18 | than 24 | than 30 | than 30 | Sum |
| Low risk | 8 (27.6%) | 16 (55.2%) | 2 (6.9%) | 3 (10.3%) | 0 | 29 (63%) |
| Intermediate to | 0 | 2 (11.8%) | 6 (35.3%) | 6 (35.3%) | 3 (17.6%) | 17 (37%) |
| high risk | | | | | | |
| Sum | 8 (17.4%) | 18 (39.1%) | 8 (17.4%) | 9 (19.6%) | 3 (6.5%) | 46 |

TABLE 9

| | |
|---|---|
| Chi-square | 23.347 |
| DF | 4 |
| Significance | P < 0.0001 |

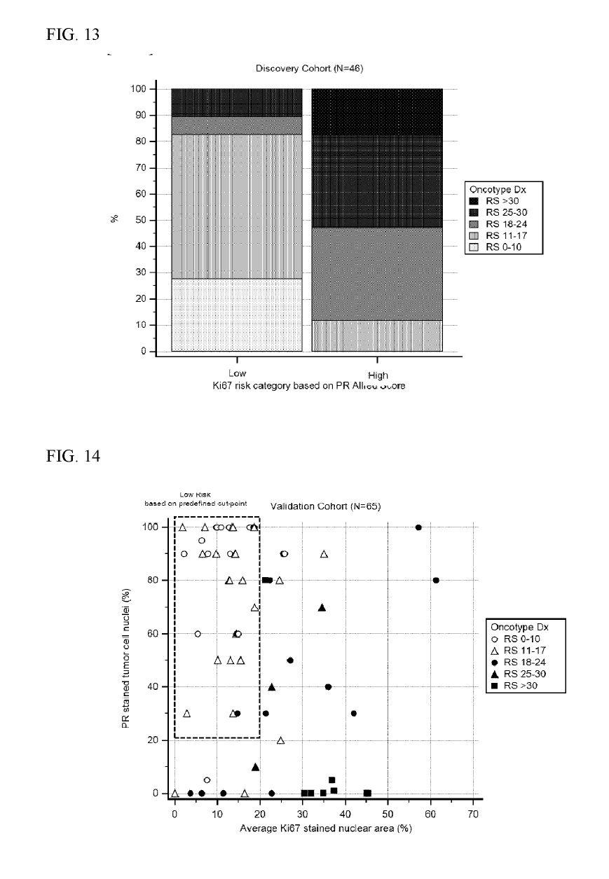

In addition, in order to predict the effect of the experimental results on clinical practice, the experimental results were graphically plotted in FIGS. 12 and 13.

As can be seen in Tables 8 and 9 above and FIGS. 12 and 13, there is a statistically very significant correlation between risk classification according to the method of the present invention and TAILROx-based Oncotype Dx classification. Namely, it can be seen that, according to the method of the present invention, three patients who must receive chemotherapy were all not classified as low risk, and low-risk patients with an RS lower than 11, who do not require chemotherapy, were all classified as low risk.

### Experimental Example 3

In order to verify whether the same results can be obtained from an independently proven cohort, the method for determining prognosis according to the present invention was additionally performed on 65 patients who underwent Oncotype Dx among patients who received treatment in Gangnam Severance Hospital. Namely, image analysis for the results of progesterone receptor and Ki67 immunostaining in breast tissue samples extracted from the 65 patients was performed using Image J program and ImmunoRatio plug in.

Specifically, Ki67 protein in each of breast tissue samples extracted from the breast cancer patients was immunostained, and the percentage of the number of cancer cells including stained Ki67 protein relative to the total number of cancer cells was selected as the X-axis variable. Furthermore, progesterone receptor was immunostained, and the percentage of the number of cancer cells including stained progesterone receptor relative to the total number of cancer cells was selected as the Y-axis variable. The percentages were graphically plotted in FIG. 14 according to different Oncotype Dx recurrence score (RS) ranges of the patients.

As can be seen in FIG. 14, in cases in which the percentage of the number of cancer cells including stained progesterone receptor relative to the total number of cancer cells after immunostaining of progesterone receptor was higher than 20% and in which the percentage of the number of cancer cells including stained Ki67 protein relative to the total number of cancer cells after immunostaining of Ki67 protein was lower than 20%, these cases all belong to the RS low-risk group.

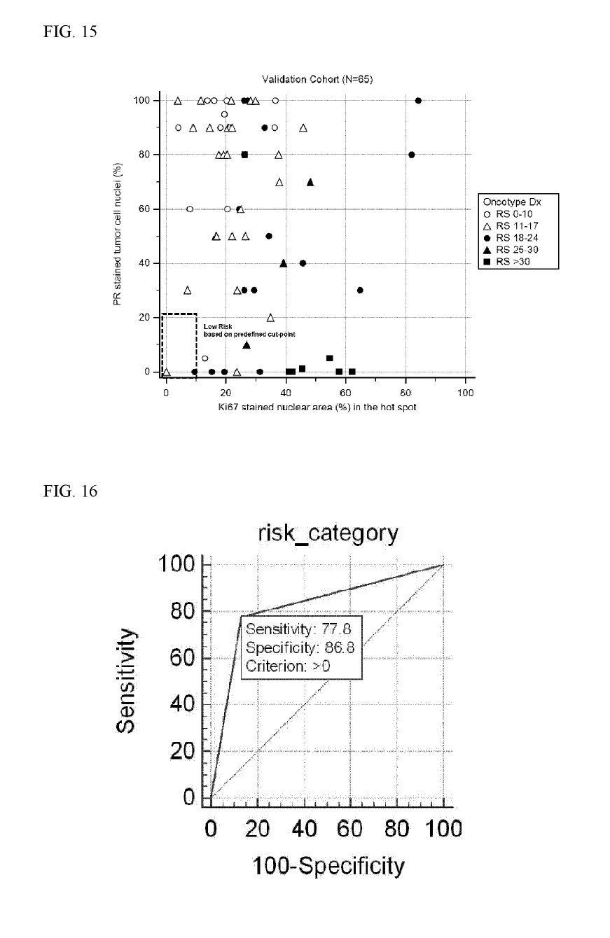

Furthermore, Ki67 protein in each of breast tissue samples extracted from the patients was immunostained, and then a spot with the highest staining index was observed with a 400× microscope, and a circular spot having a radius of about 400 to 650 μm was selected as a hot spot. For the hot spot, the percentage of the number of cancer cells including stained Ki67 protein relative to the total number of cancer cells was selected as the X-axis variable, and the percentage of the number of cancer cells including stained progesterone receptor relative to the total number of cancer cells was selected as the Y-axis variable, and the percentages were graphically plotted in FIG. 15 according to different Oncotype Dx RS ranges of the patients.

As can be seen in FIG. 15, in cases in which the percentage of the number of cancer cells including stained progesterone receptor relative to the total number of cancer cells after immunostaining of progesterone receptor was 20% or lower and in which the percentage of the number of cancer cells including stained Ki67 protein relative to the total number of cancer cells after immunostaining of Ki67 protein was lower than 10%, these cases mostly belong to the RS low-risk group.

In order to verify how the results obtained according to the method of the present invention would accurately predict the RS category of Oncotype Dx, the results of risk classification performed using the results of progesterone receptor immunostaining and Ki67 immunostaining as shown in FIGS. 14 and 15 were compared with the results of Oncotype Dx classification. The results of the comparison are shown in Table 10 below.

TABLE 10

| | Oncotype Dx classification | | |
|---|---|---|---|
| Classification | | Intermediate to | |
| according to the | Low risk (RS | high risk (RS of | |
| present invention | lower than 18) | 18 or higher) | Sum |
| Low risk | 33 (86.5%) | 6 (13.5%) | 37 (56.9%) |
| Intermediate | 5 (21.4%) | 21 (78.6%) | 28 (43.1%) |
| to high risk | | | |
| Sum | 38 (58.5%) | 27 (41.5%) | 65 |

(p=0.000000303 (Fisher's exact test))

As can be seen in Table 10 above, there was a very significant correlation between classification according to the method of the present invention and Oncotype Dx classification (p=0.000000303 (Fisher's exact test)), and about 83% ((23+18)/65*100) in the method of the present invention was identical to that in Oncotype Dx classification.

In addition, in order to confirm the statistical accuracy of the results, ROC (Receiver-Operating Characteristic) analysis was additionally performed. The results of the analysis are shown in Table 11 below and FIG. 16.

TABLE 11

| | | |
|---|---|---|
| Sensitivity | 77.78% | 57.74% to 91.38% |
| Specificity | 86.84% | 71.91% to 95.59% |
| Area Under Curve | 0.82 | 0.71 to 0.91 |
| Positive likelihood ratio | 5.91 | 2.55 to 13.71 |
| Negative likelihood ratio | 0.26 | 0.12 to 0.52 |
| Disease prevalence | 41.54% | 29.44% to 54.44% |
| Positive predictive value | 80.77% | 60.65% to 93.45% |
| Negative predictive value | 84.62% | 69.47% to 94.14% |

As can be seen in Table 11 above and FIG. 16, sensitivity was 77.78% and specificity was 86.84%, suggesting that intermediate- or high-risk patients can be identified.

Furthermore, Table 12 below compares the results of risk classification, performed using the results of progesterone receptor immunostaining and Ki67 immunostaining as shown in FIGS. 14 and 15, with the results of TAILORx-based Oncotype Dx classification.

TABLE 12

| | TAILORx-based Oncotype Dx classification | | | | | |
|---|---|---|---|---|---|---|
| Classification | | | RS higher | RS higher | | |
| according to the | | | than 18 but | than 24 but | | |
| present | RS lower | RS of 11 to | not higher | not higher | RS higher | |
| invention | than 11 | 18 | than 24 | than 30 | than 30 | Sum |
| Low risk | 12 (30.8%) | 21 (53.8%) | 5 (12.8%) | 1 (2.6%) | 0 | 39 (60.0%) |
| Intermediate to | 1 (3.8%) | 4 (15.4%) | 11 (42.3%) | 2 (7.7%) | 8 (30.8%) | 26 (40.0%) |
| high risk | | | | | | |
| Sum | 13 (20%) | 25 (38.5%) | 16 (24.6%) | 3 (4.6%) | 8 (12.3%) | 65 |

(p=0.00000082 (Fisher's exact test))

As can be seen in Table 12 above, the method of the present invention can very accurately identify low-risk patients who do not require chemotherapy.

Furthermore, in order to confirm the statistical significance of the experimental results, Chi-squared test was performed. As a result, as can be seen in Table 13 below, the results are statistically significant.

TABLE 13

| | |
|---|---|
| Chi-square | 30.053 |
| DF | 4 |
| Significance | P < 0.0001 |

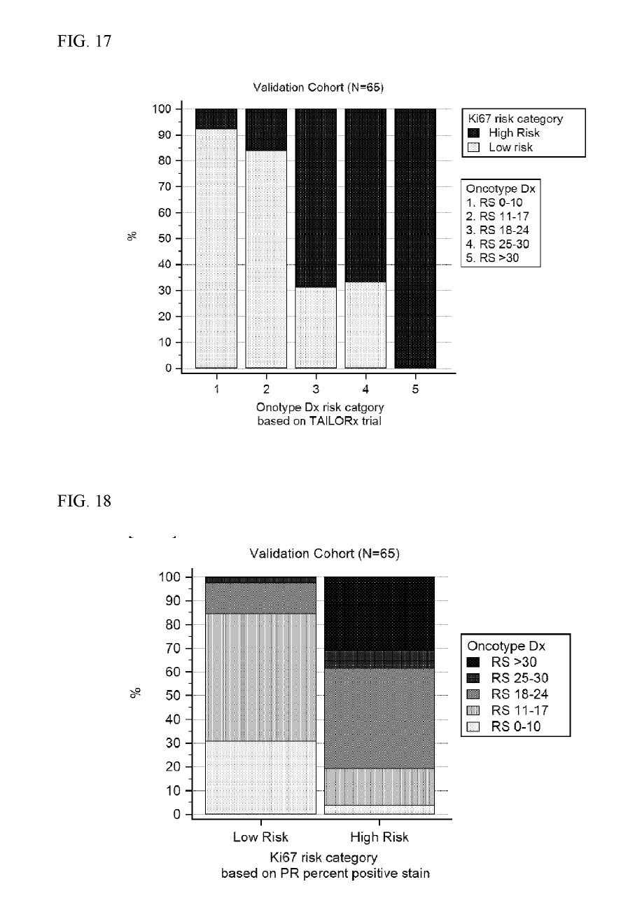

In addition, in order to predict the effect of the experimental results on clinical practice, the experimental results were graphically plotted in FIGS. 17 and 18. Among the patients classified as low risk by the method of the present invention, there was no patient with an Oncotype Dx RS higher than 30, who necessarily require chemotherapy. Therefore, it can be seen that the method of the present invention can statistically significantly or clinically very significantly predict the risk category of Oncotype Dx.

### Experimental Example 4

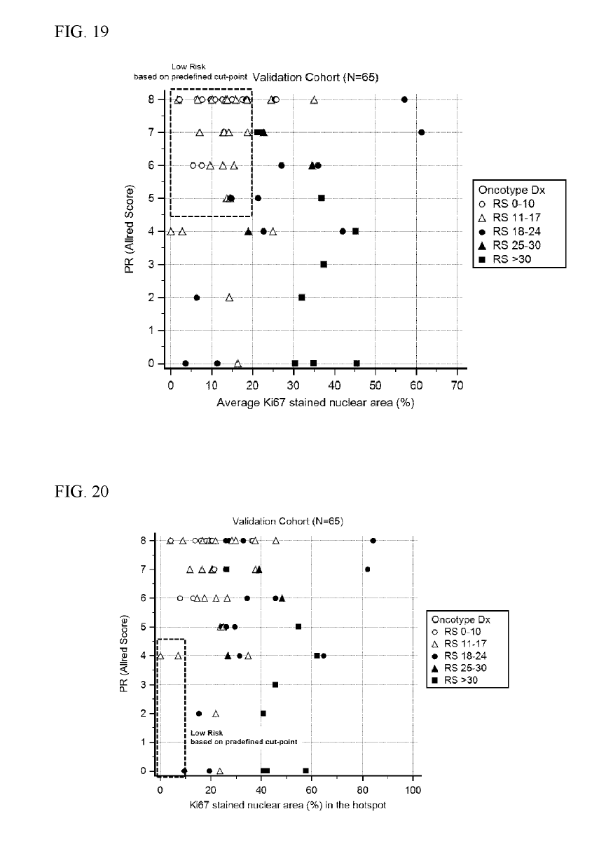

For 65 patients who underwent Oncotype Dx among patients who received treatment in Gangnam Severance Hospital as described in Experimental Example 3, an Allred score after immunostaining of progesterone receptor was measured and selected as the Y-axis variable. Furthermore, Ki67 protein in each of breast tissue samples extracted from the patients was immunostained, and the percentage of the number of cancer cells including stained Ki67 protein relative to the total number of cancer cells was selected as the X-axis variable. Then, the Allred score and the percentage were graphically plotted in FIG. 19 according to different Oncotype Dx RS ranges of the patients.

As can be seen in FIG. 19, in cases in which the Allred score after immunostaining of progesterone receptor was 5 or higher and in which the percentage of the number of cancer cells including stained Ki67 protein relative to the total number of cancer cells after immunostaining of Ki67 protein was lower than 20%, these cases mostly belong to the RS low-risk group.

Furthermore, Ki67 protein in each of breast tissue samples extracted from the patients was immunostained, and then a spot with the highest staining index was observed with a 400× microscope, and a circular spot having a radius of about 400 to 650 μm was selected as a hot spot. For the hot spot, the percentage of the number of cancer cells including stained Ki67 protein relative to the total number of cancer cells was selected as the X-axis variable, and the Allred score after immunostaining of progesterone receptor was selected as the Y-axis variable, and the percentage and the Allred score were graphically plotted in FIG. 20 according to different Oncotype Dx RS ranges of the patients.

As can be seen in FIG. 20, in cases in which the Allred score after immunostaining of progesterone receptor was lower than 5 and in which the percentage of the number of cancer cells including stained Ki67 protein relative to the total number of cancer cells after immunostaining of Ki67 protein was lower than 10%, these cases all belong to the RS low-risk group.

Therefore, it can be seen that, when the method of the present invention is used, the prognosis of breast cancer patients can be predicted using the Allred score and the results of Ki67 protein immunostaining in a very simple manner or with high accuracy, and particularly when Allred score is lower than 5, the accuracy of the prediction can further be increased by selecting and analyzing a hot spot.

Table 14 below compares the results of risk classification shown in FIGS. 19 and 20 and the results of Oncotype Dx classification.

TABLE 14

| | Oncotype Dx classification | | |
|---|---|---|---|
| Classification | | Intermediate to | |
| according to the | Low risk (RS | high risk (RS of | |
| present invention | lower than 18) | 18 or higher) | Sum |
| Low risk | 32 (86.5%) | 5 (13.5%) | 37 (56.9%) |
| Intermediate | 6 (21.4%) | 22 (78.6%) | 28 (43.1%) |
| to high risk | | | |
| Sum | 38 (58.5%) | 27 (41.5%) | 65 |

(p=0.000004738 (Fisher's exact test))

As can be seen in Table 14 above, the method of the present invention can accurately identify low-risk patients who do not require chemotherapy. Since the low-risk group accounts for 50% or higher, many patients can be liberated from unnecessary chemotherapy, and thus the psychological anxiety of the patients and the physical burden and economic burden caused by chemotherapeutic side effects can be alleviated.

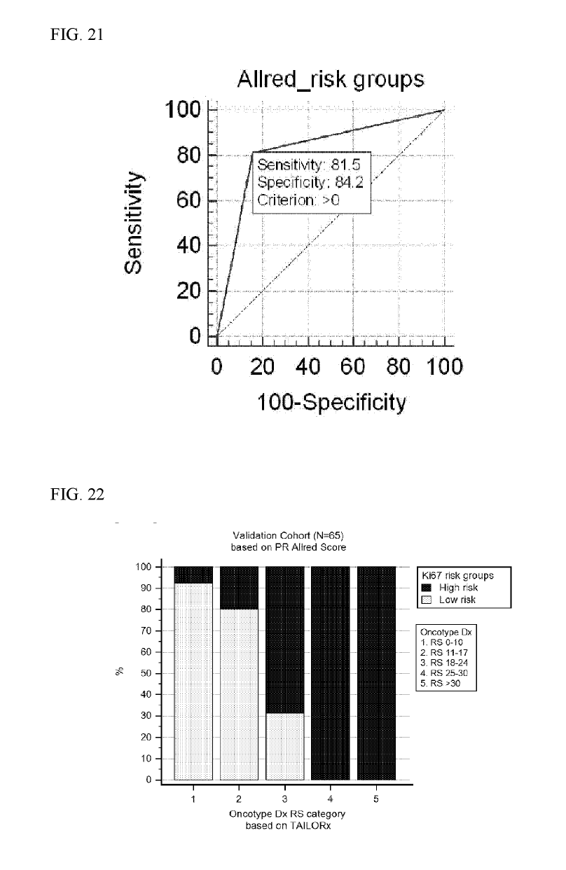

In addition, in order to confirm the statistical accuracy of the results, ROC (Receiver-Operating Characteristic) analysis was additionally performed. The results of the analysis are shown in Table 15 below and FIG. 21.

TABLE 15

| | | |
|---|---|---|
| Sensitivity | 81.48% | 61.92% to 93.70% |
| Specificity | 84.21% | 68.75% to 93.98% |
| Area Under Curve | 0.83 | 0.71 to 0.91 |
| Positive likelihood ratio | 5.16 | 2.42 to 10.99 |
| Negative likelihood ratio | 0.22 | 0.10 to 0.49 |
| Disease prevalence | 41.54% | 29.44% to 54.44% |
| Positive predictive value | 78.57% | 59.05% to 91.70% |
| Negative predictive value | 86.49% | 71.23% to 95.46% |

As can be seen in Table 15 above and FIG. 21, sensitivity was 81.48% and specificity was 84.21%, suggesting that intermediate- or high-risk patients can be identified.

Furthermore, Table 16 below compares the results of risk classification shown in FIGS. 19 and 20 with the results of TAILORx-based Oncotype Dx classification. In order to confirm the statistical significance of the experimental results, Chi-squared test was performed, and the results are shown in Table 17 below.

TABLE 16

| | TAILORx-based Oncotype Dx classification | | | | | |
|---|---|---|---|---|---|---|
| Classification | | | RS higher | RS higher | | |
| according to the | | | than 18 but | than 24 but | | |
| present | RS lower | RS of 11 to | not higher | not higher | RS higher | |
| invention | than 11 | 18 | than 24 | than 30 | than 30 | Sum |
| Low risk | 12 (32.4%) | 20 (54.1%) | 5 (13.5%) | 0 | 0 | 37 (56.9%) |
| Intermediate to | 1 (3.6%) | 5 (17.9%) | 11 (39.3%) | 3 (10.7%) | 8 (28.6%) | 28 (43.1%) |
| high risk | | | | | | |
| Sum | 13 (20%) | 25 (38.5%) | 16 (24.6%) | 3 (4.6%) | 8 (12.3%) | 65 |

TABLE 17

| | |
|---|---|
| Chi-square | 30.904 |
| DF | 4 |
| Significance | P < 0.0001 |

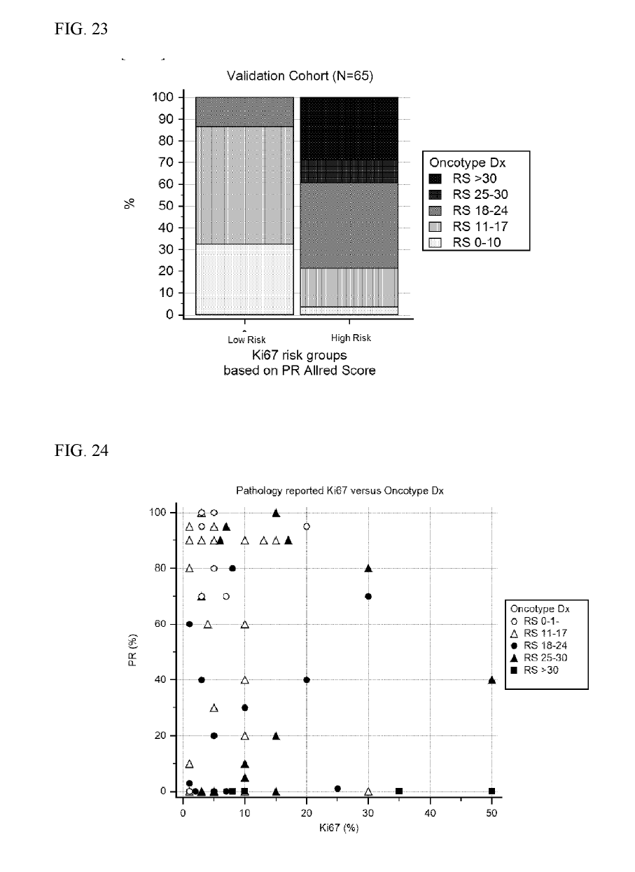

In addition, in order to predict the effect of the experimental results on clinical practice, the experimental results were graphically plotted in FIGS. 22 and 23.

As can be seen in Tables 16 and 17 above and FIGS. 22 and 23, there is a statistically very significant correlation between risk classification according to the present invention and TAILROx-based Oncotype Dx classification. Namely, among the patients classified as low risk by the method of the present invention, there was no high-risk patient who necessarily requires chemotherapy, like Oncotype Dx or TAILORx.

### Comparative Example 1

Analysis of the clinical results of Ki67 immunostaining was relied on judgment of the pathologist without performing image analysis as described in Experimental Example 1. In this case, as shown in FIG. 24, when only the results of Ki67 or progesterone receptor immunostaining were used, it was impossible to achieve RS risk classification or prognostic prediction.

Specifically, in the case in which patients are classified as low risk when the percentage of the number of cancer cells including stained Ki67 protein relative to the total number of cancer cells after immunostaining of Ki67 protein is lower than 20%, it can be seen that several high-risk patients are wrongly classified as low risk. Furthermore, in the case in which patients are classified as high risk when the percentage of the number of cancer cells including stained Ki67 protein is lower than 10%, it can be seen that several low-risk patients are wrongly classified as high-risk patients.

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereof.

### INDUSTRIAL APPLICABILITY

The present invention relates to an apparatus and a method for determining breast cancer prognosis and whether to use chemotherapy, and more particularly to an apparatus and method capable of predicting prognosis and chemotherapeutic effects, which are necessary in determining a treatment plan for a breast cancer patient, based on the results of immunostaining of progesterone receptor and the results of immunostaining of Ki67 protein.

What is claimed is:

1. An apparatus for determining breast cancer prognosis and whether to use chemotherapy, the apparatus comprising:
a first input unit configured to receive a percentage of number of cancer cells including stained progesterone receptor relative to total number of cancer cells or an Allred score after immunostaining of progesterone receptor in a breast tissue sample extracted from a breast cancer patient;
a second input unit configured to receive a percentage of number of cancer cells including stained Ki67 protein relative to total number of cancer cells after immunostaining of Ki67 protein in the breast tissue sample;
a classification calculation unit configured to classify the breast cancer patient as low risk when:
i. the percentage of number of cancer cells including stained progesterone receptor relative to total number of cancer cells, inputted in the first input unit, is higher than 20%, or the Allred score is 5 or higher, and when the percentage of number of cancer cells including stained Ki67 protein relative to total number of cancer cells, inputted in the second input unit, is lower than 20%; or
ii. the percentage of number of cancer cells including stained progesterone receptor relative to total number of cancer cells, inputted in the first input unit, is 20% or lower, or the Allred score is lower than 5, and when the percentage of number of cancer cells including stained Ki67 protein relative to total number of cancer cells, inputted in the second input unit, is lower than 10%; and
an output unit configured to output analysis result from the classification calculation unit.

2. The apparatus of claim 1, wherein the percentage of number of cancer cells including stained Ki67 protein after immunostaining of Ki67 protein, which is inputted in the second input unit, is a result obtained for a circular spot with the highest staining index, which has a radius of 400 to 650 after immunostaining.

3. The apparatus of claim 1, wherein the low risk corresponds to a recurrence score (RS) of 0 to 18 as determined by Oncotype Dx.

4. (canceled)

5. (canceled)

6. (canceled)

7. A method for providing information for determining breast cancer prognosis and whether to use chemotherapy, the method comprising classifying a breast cancer patient as low risk when a percentage of number of cancer cells including stained progesterone receptor relative to total number of cancer cells after immunostaining of progesterone receptor in a breast tissue sample extracted from the breast cancer patient is higher than 20%, or an Allred score after immunostaining of progesterone receptor in the breast tissue sample is 5 or higher, and when a percentage of number of cancer cells including stained Ki67 protein relative to total number of cancer cells after immunostaining of Ki67 protein is lower than 20%.

8. The method of claim 7, wherein the percentage of number of cancer cells including stained Ki67 protein after immunostaining of Ki67 protein in the breast tissue sample is a result obtained for a circular spot with the highest staining index, which has a radius of 400 to 650 after immunostaining.

9. The method of claim 7, wherein the low risk corresponds to a recurrence score (RS) of 0 to 18 as determined by Oncotype Dx.

10. A method for providing information for determining breast cancer prognosis and whether to use chemotherapy, the method comprising classifying a breast cancer patient as low risk when a percentage of number of cancer cells including stained progesterone receptor relative to total number of cancer cells after immunostaining of progesterone receptor in a breast tissue sample extracted from the breast cancer patient is 20% or lower, or an Allred score after immunostaining of progesterone receptor in the breast tissue sample is lower than 5, and when a percentage of number of cancer cells including stained Ki67 protein relative to total number of cancer cells after immunostaining of Ki67 protein in the breast tissue sample is lower than 10%.

11. The method of claim 10, wherein the percentage of number of cancer cells including stained Ki67 protein after immunostaining of Ki67 protein in the breast tissue sample is a result obtained for a circular spot with the highest staining index, which has a radius of 400 to 650 after immunostaining.

12. The method of claim 10, wherein the low risk corresponds to a recurrence score (RS) of 0 to 18 as determined by Oncotype Dx.
